(19) United States
(12) Patent Application Publication
(10) Pub. No.: US 2025/0010096 A1
(43) Pub. Date: Jan. 9, 2025

(54) SYSTEM AND PROCESS TO TREAT BRAIN DISEASE USING RESONANCE ENTRAINMENT OF ACTION WAVES

(71) Applicants: Raymond Stanley Kasevich, Mt. Washington, MA (US); Peter McEachern, Goshen, CT (US)

(72) Inventors: Raymond Stanley Kasevich, Mt. Washington, MA (US); Peter McEachern, Goshen, CT (US)

(21) Appl. No.: 18/763,260

(22) Filed: Jul. 3, 2024

Related U.S. Application Data
(60) Provisional application No. 63/511,698, filed on Jul. 3, 2023.

(51) Int. Cl. A61N 5/06; A61H 23/00; A61N 2/00

(52) U.S. Cl. CPC A61N 5/0622; A61H 23/00; A61N 2/006; A61N 2005/0663

(57) ABSTRACT

A system and process for the treatment of brain disease by the generation of sensory activities of sound from music, blinking light of a specific color and intensity, and electromagnetic fields from a source such as a pulsating magnetic field. The energy of the sensory activities may be employed to selectively excite brain neuron action potentials. The action potentials can create compressive and expansive forces acting direct on any diseased plaque buildup on the surface membrane of the neurons to cause reduction in volume of the diseased plaque such as associated with Alzheimer's Disease. When a resonant vibration of action potentials exists, the volume reduction of plaque will be increased because of the increase in the strength of action potentials at resonant frequencies thereby reducing the amount of the toxic plaque buildup associated with the diseased neuron.

### STATEMENT REGARDING FEDERALLY SPONSORED RESEARCH OR DEVELOPMENT

Not applicable.

### COPYRIGHT NOTICE

A portion of this disclosure contains material which is subject to copyright protection. The copyright owner has no objection to the photocopy reproduction by anyone of the patent document or the patent disclosure in exactly the form it appears in the Patent and Trademark Office patent file or records, but otherwise reserves all copyright rights whatsoever. 37 C.F.R 1.71(d).

### BACKGROUND OF THE INVENTIVE CONCEPT

### 1. Field of the Invention

The present inventive concept relates to a process to treat brain disease using resonance entrainment of action waves. More particularly, but not exclusively, this inventive concept relates to a process to treat brain disease by applying resonant electrostatic force waves of an action potential that achieves mechanical displacement, separation, and/or breakup of plaque material in the brain.

### SUMMARY OF THE INVENTIVE CONCEPT

The present general inventive concept provides a process to treat brain disease by applying resonant electrostatic force waves of an action potential that achieves mechanical displacement, separation, and/or breakup of plaque material in the brain.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a generator of music having the appropriate frequency components to excite the resonant action potentials in the cerebral cortex for compression of the diseased plaque associated brain neurons.

In an exemplary embodiment, the appropriate frequencies are applied and positioned outside the brain to achieve an overall sensory activity for enhancement of the treatment volume of diseased brain neurons.

In an exemplary embodiment, reduction of brain AD is detected by TMS measurement of the eye retina.

The foregoing and/or other features and utilities of the present general inventive concept may also be achieved by providing a generator to generate a blinking light for a human eye in the visual spectrum of appropriate color, intensity and pulse rate to excite the resonant action potentials in the cerebral cortex for the compression of the diseased plaque associated with brain neurons.

The foregoing and/or other features and utilities of the present general inventive concept may also be achieved by providing a generator of electromagnetic waves over a broad range of oscillations that can include electric or magnetic energy separately applied such as with an antenna or commercially available TMS device (trans magnetic stimulation) to excite the resonant action potentials in the cerebral cortex for the compression of the diseased plaque associated with brain neurons.

### BRIEF DESCRIPTION OF THE DRAWINGS

These and/or other features and utilities of the present inventive concept will become apparent and more readily appreciated from the following description of the embodiments, taken in conjunction with the accompanying drawings of which:

FIG. 1 illustrates a result of the simulation where iterative calculations reveal an orderly relationship between peak frequency and outward potassium conductance;

FIG. 2 illustrates the stability of the peak resonance frequency during repeated cycling of pulses within the corticothalamic loop circuit;

FIG. 3 illustrates music specifically designed for a 40 Hertz frequency spectrum to treat brain disease, according to an example embodiment of the present inventive concept;

FIG. 4 illustrates a Muse EEG Headband used to read EEG brain frequencies in four general brain regions while applying the music illustrated in FIG. 3;

FIG. 5 illustrates regions of the brain in which the EEG measurements are obtained using the Muse EEG Headband illustrated in FIG. 4;

FIG. 6 illustrates a graph of frequency vs. time where brain waves are detected in response to music played in the 40 Hertz frequency spectrum;

FIG. 7 illustrates observations of neural firing (Action Potentials) from a 60-80 Hz session at regular intervals using an EEG and Lab scribe v4 software; and

FIG. 8 illustrates observations of neural firing (Action Potentials) from a 40 Hz session at regular intervals using an EEG and Lab scribe v4 software.

The drawings illustrate a few exemplary embodiments of the present inventive concept, and are not to be considered limiting in its scope, as the overall inventive concept may admit to other equally effective embodiments. The elements and features shown in the drawings are to scale and attempt to clearly illustrate the principles of exemplary embodiments of the present inventive concept. In the drawings, reference numerals designate like or corresponding, but not necessarily identical, elements throughout the several views.

### DETAILED DESCRIPTION OF THE PREFERRED EMBODIMENTS

Reference will now be made in detail to the embodiments of the present general inventive concept, examples of which are illustrated in the accompanying drawings, wherein like reference numerals refer to the like elements throughout. The embodiments are described below in order to explain the present general inventive concept while referring to the figures. Also, while describing the present general inventive concept, detailed descriptions about related well-known functions or configurations that may diminish the clarity of the points of the present general inventive concept are omitted.

It will be understood that although the terms “first” and “second” are used herein to describe various elements, these elements should not be limited by these terms. These terms are only used to distinguish one element from another element. Thus, a first element could be termed a second element, and similarly, a second element may be termed a first element without departing from the teachings of this disclosure.

Expressions such as “at least one of,” when preceding a list of elements, modify the entire list of elements and do not modify the individual elements of the list.

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the detailed description of the invention. Thus, the terms used herein have to be defined based on the meaning of the terms together with the description throughout the specification.

Also, when a part “includes” or “comprises” an element, unless there is a particular description contrary thereto, the part can further include other elements, not excluding the other elements. In the following description, terms such as “unit” and “module” indicate a unit to process at least one function or operation, wherein the unit and the module may be embodied as hardware or software or embodied by combining hardware and software.

Hereinafter, one or more exemplary embodiments of the present general inventive concept will be described in detail with reference to accompanying drawings.

Exemplary embodiments of the present general inventive concept are directed to a system and method to treat brain disease using resonance entrainment of action waves. More particularly, but not exclusively, this inventive concept relates to a system and method to treat brain disease by applying resonant electrostatic force waves of an action potential that achieves mechanical displacement, separation, and/or breakup of plaque material in the brain.

Resonant conditions of apical dendrites (a dendrite that emerges from the apex of a pyramidal cell in the cortices) have been theoretically demonstrated to depend on the outgoing potassium leak currents and membrane potential with the oscillation frequency bandwidth approaching one Hertz (Hz) over a frequency range of 20 to 80 Hz from thalamocortical looping currents (currents of bidirectional communication between the neocortex and the thalamus). Many studies have shown that mechanical displacement of the axonal membrane (a surface membrane of the axonal process from the point at which it emerges from the axon hillock of the neuronal perikaryon to the axonal terminal or nerve ending, which forms a synapse on another cell) accompanies the electrical pulse defining an action potential (AP). Action potentials are sufficient in electrical intensity in membrane systems to mechanically deform and compress plaque or myelin during the depolarization phase of the action potential (AP).

For many decades action potentials (APs) have been measured using electrophysiological methods and have been understood as being electrical signals generated and propagated along the axonal membrane. Experimental studies have shown that the AP is accompanied by fast and temporary mechanical changes. These changes include changes in the axonal radius, pressure, optical properties, and the release and subsequent absorption of a small amount of heat. However, despite the amount of experimental evidence up to the present time, the physical basis for the mechanical and thermal signals that accompany the AP remains poorly understood. In fact, there appears to have been no attempts to quantitatively describe the mechanical component of the AP as an electrically driven phenomenon. In the present inventive concept as described herein, a multilayer, cylindrical model of the neuron as an elastic and dielectric tube filled and surrounded with viscous fluid is considered. As the action potential (AP) propagates along an axonal membrane, electric forces from the AP act on microscopic electric dipoles that constitute the electrical properties of the membrane as a dielectric medium. These forces lead to co-propagating waves of electrical (AP) and mechanical energy or action wave (AW) both inside and outside axions.

Recent studies have shown that compression or reduction in the lipid bilayer thickness regulates the aggregation and cytotoxicity of Amyloid-β (Aβ) plaque as a mechanism by which the Aβ plaque exerts its toxicity in Alzheimer's disease.

In the present inventive concept, the production of action potentials (AP) from resonant oscillations has shown to provide compressive forces on thin layers of plaque in a multiple layer dielectric model of the myelin layer or sheath (an insulating layer or sheath that forms around nerves, including those in the brain and spinal cord and allows electrical impulses to transmit quickly and efficiently along the nerve cells). Calculations also show these forces to be in the same range as to affect movements of biological tissue. Accordingly, the present inventive concept focuses on controlling the cytotoxicity of Amyloid-β (Aβ) and other plaque diseases through a cognitive process involving the production of action potentials (AP) from apical dendrite resonant oscillations sufficient in intensity to control the thickness of plaque aggregates by electrically driven (AP) forces or action waves on dielectric materials such as Aβ plaque.

The foundation of the present inventive concept, the cognitive treatment of diseased neurons, is based on a combination of: (1) electric resonance theory: specific frequencies of a sensory stimulus such as sound, light, music, etc., that support the creation of frequency selective brain oscillations which can be finely tuned to a narrow band of frequencies at resonance for the production of action potentials (AP) of appropriate intensity and timing; and (2) mechanical pulses or action waves (AW) or vibrations on the membrane of the neurons. The resulting action potentials will produce a mechanical pulse that propagates synchronously with the action potential pulse. The resultant electrostatic forces produce an electric field stress in the plaque attached to the myelin sheath and will result generally in compressive forces which can cause plague reduction, as recent MIT results have demonstrated using light flicker at 40 Hz. Optically driving fast-spiking neurons at gamma (40 Hz), but not other frequencies, reduces levels of forms of amyloidal-β (Aβ) plaque using a 40 Hz light-flickering regime in the visual cortex of pre-depositing mice and mitigated plaque load in aged, depositing mice.

In this invention it has been determined that bundles of long apical dendrites dominating the neurons, which make up the cerebral cortex, can produce sustained oscillations at a specific resonant frequency in thalamocortical loops. These sustained oscillations of electric charge in apical dendrites produce synchronous pulse outputs in circuit loops containing apical dendrites when the loops show oscillatory activity and pulse surges of electric current repeatedly pass along the membrane. After each pass along the dendrite the electric charge oscillation frequencies begin a tuning process much as in a radio receiver when the dial is set for the desired frequency. After many cycles of charge surges in the dendrite from beginning to end, the frequency of surges become finely tuned. For example, after eight loops or cycles the profile or amplitude is sharpened to a frequency width of less than 1 Hz with the peak frequency of 40 Hz in the gamma band of possible frequencies. The resultant action potentials (AP) will be of sufficient electric field intensity within the membranes and the membrane surfaces to mechanical deformation and compress thin plaque on the myelin sheaths during the depolarization phase of the action potential (AP).

The existence of transient mechanical changes in the cell cortex has been confirmed by micropipette aspiration experiments. The physical reason for this process is the fact that action potentials (AP) create much stronger electric field intensities in the myelin sheath as compared to the electric fields outside in fluids. The membrane (myelin sheath) acts essentially as a waveguide for the pulse of electrostatic AP energy because the highly conductive boundaries surrounding the myelin sheath forces most of the electric field energy of the action potential (AP) to reside within the myelin sheath. These action potential (AP) electric fields will produce various mechanical forces on thin membrane surfaces, such as on and within Aβ plaque on the myelin sheath, because of the microscopic electric dipoles that give the membrane its dielectric properties.

When the corticothalamic loop shows oscillatory activity, pulse surges of current repeatedly pass along the membrane. After each complete pass along the apical dendrite, the resonance profile narrows only slightly so that many cycles of surges are needed to sharpen the profile to the point where the frequency of surges are fine-tuned close to one specific frequency. As the pulse-surges move repeatedly down the apical dendrite the outflow of potassium ions changes the shape of the resonance profile, compartment after compartment. The narrowing of the profile comes about by increasing the occurrences of specific resonance frequencies near the peak resonance frequency region of the profile and decreasing the occurrences of specific resonance frequencies remote from the peak frequency.

According to an example embodiment of the present inventive concept, four equally spaced frequencies in the 0-100 Hz range were selected, 20, 40, 60, and 80 Hz, and the outward flow of potassium ions, gᵣ, was calculated for each frequency using the following Equation (1):

$${E}_{2} ( ω ) / \text{?} ( ω ) = \frac{k ( s + \text{?} )}{( s + {ω}_{2} ) ( s + {ω}_{3} )} , \qquad ( 1 )$$

$$where$$

$$s = j ω , \qquad ( 2 )$$

$${ω}_{1} = {g}_{s} / {C}_{s} , \qquad ( 3 )$$

$${ω}_{2} = {g}_{r} + 2 {g}_{s} / {C}_{r} = {C}_{s} , \qquad ( 4 )$$

$${ω}_{1} = {g}_{r} / {C}_{r} , \qquad ( 5 )$$

$$and$$

$$k = \text{?} / ( {C}_{r} + 2 {C}_{s} ) ( \text{?} ) , \qquad ( 6 )$$

$$\text{?} \text{indicates text missing or illegible when filed}$$

(Cₛ non-linear surface capacitance, Cᵣ is horizontal capacitance, gₛ is horizontal conductance, E₂(ω)/I_{I}(ω) is the impedance transfer function, k is a constant, ω₁, ω₂, and ω₃ have units of radian frequency and are referred to here as relaxation frequencies, following classical circuit theory concepts. The relaxation frequencies and k completely characterize the bandpass frequency behavior of the dendrite model)
   which is based on a single compartment. Iterative calculations converged to a value of gᵣ that maximized the energy transfer from one compartment to the next compartment. Appropriate geometric parameters of the underlying leaky cable theory and electrical parameters of the membrane were obtained from “Theory of Electric Resonance in the Neocortical Apical Dendrite,”(https://journals.plos.org/plosone/article/file?id=10.1371/journal.pone. 0023412&type=printable), by Ray S. Kasevich and David LeBerge, and entered into the following Equation (12) defining the transfer impedance:

$${E}_{2} ( ω ) / \text{?} ( ω ) = \frac{k \sqrt{( {ω}^{2} + {ω}_{1}^{2}}}{\sqrt{( {ω}^{2} + {ω}_{2}^{2} )} \sqrt{( {ω}^{2} + {ω}_{3}^{2} )}} , \qquad ( 12 )$$

$$\text{?} \text{indicates text missing or illegible when filed}$$

(ω₁, ω₂, and ω₃ and k are defined by Equations (3-6 above))
   of “Theory of Electric Resonance in the Neocortical Apical Dendrite,”(https://journals.plos.org/plosone/article/file?id=10.1371/journal.pone. 0023412&type=printable), by Ray S. Kasevich and David LeBerge. The measure of energy transfer is transfer impedance.

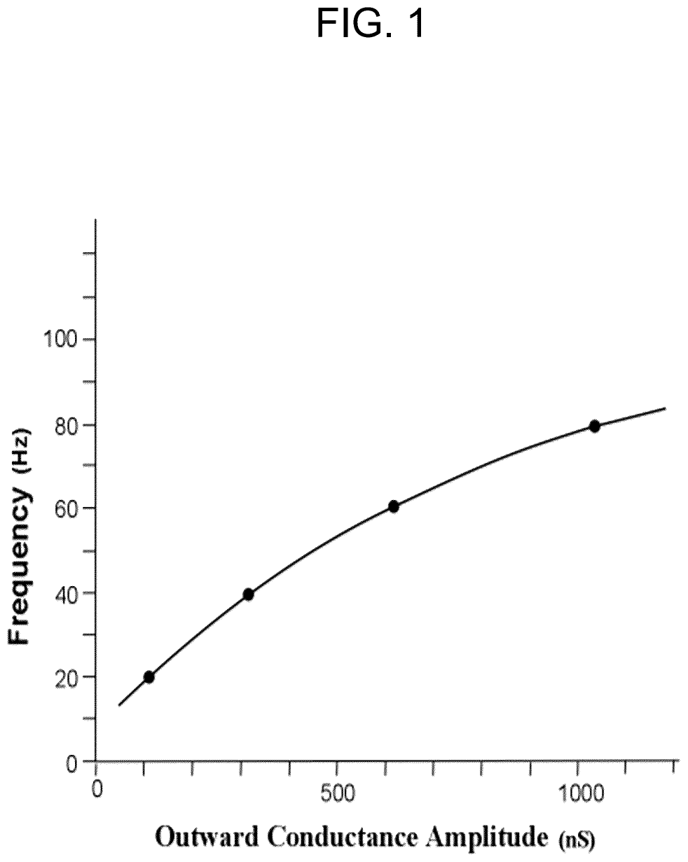

FIG. 1 illustrates a result of a simulation where iterative calculations of the outward flow of potassium ions for each frequency reveal an orderly relationship between peak frequency and outward potassium conductance, gᵣ. Here it is observed that increasing the outward conductance increases the frequency of the peak of the frequency profile, but the peak frequency approaches a limit as outward conductance is increased toward higher values. This result confirms that the peak frequency of an apical dendrite oscillation can be set by adjusting the rate of outward flow of potassium ions through the apical dendrite membrane.

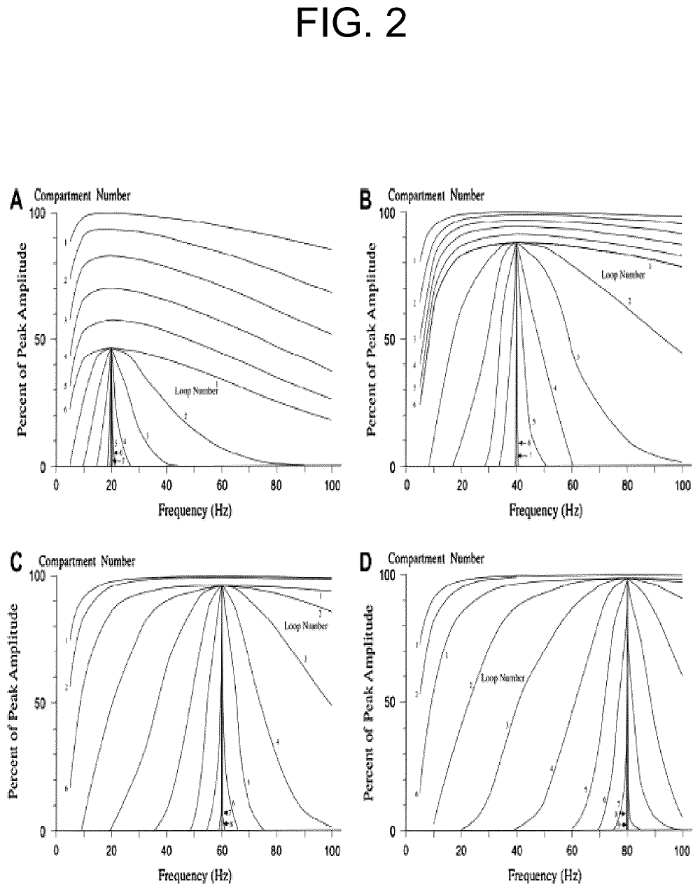

In summary, the main findings of the simulation experiments are: 1) that the relationship between outward potassium current and the peak frequency of apical dendrite oscillation (shown in FIG. 1 as a curve) is such that the peak frequency increases as outward current increases; and 2) the four sets of resonance profile curves, as illustrated in FIG. 2, show the establishment of four peak oscillation frequencies in the apical dendrite during early cycles of current in the corticothalamic loop. Moreover, these resonance profile curves show that each profile around these peak resonance frequencies is sharpened to a width of less than 1 Hz by repeated cycling of electric surges through the apical dendrite.

It should be noted that the addition of the Aβ plaque would generally lower the resonant frequencies vs. outward conductance as predicted in the present model.

Hence, it is determined here that a potential mechanism for plaque reduction can be explained by action potentials (AP) coupling with mechanical action to control lipid bilayer thickness to regulate both the aggregation and cytotoxicity in Alzheimer's (AZ). Here energy storage displacement theory is used from electrostatic dielectric theory to calculate the compressive forces. The strength of the action potential is derived from the peak values of action potentials. These electric force fields exist in dielectric materials as well as physically and electrically interacting dielectric materials such as Amyloidal plaques in various forms and myelin. Studies have been conducted on the dielectric properties of these biologic materials. The high electric conductivities of extracellular fluids and intercellular fluids create intense electric fields from action potentials that exist primarily in the low conductivity regions such as myelin and adjacent plaque materials.

To enhance the compressive force, since this force varies as the square of the amplitude of the action potential, loop resonances possible in the cerebral cortex provide insight into controlling the amplitude of the dielectric force depending on the particular resonant frequency and its associated bandwidth. A narrow band response from the thalamocortical loop circuitry may increase or decrease the compressive forces. Simulations have shown that approximately 7 loops can narrow the action potential to 1 Hz, meaning that a strong or intense action potential pulse can be possible with looping leading to more intense action potentials for plaque compression.

### Example Embodiments of the Inventive Process

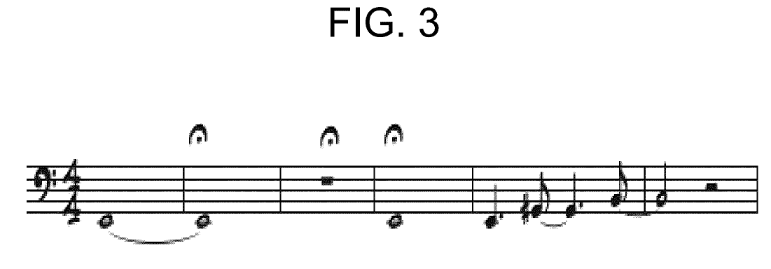

FIG. 3 illustrates a composition of music specifically designed for a 40 Hertz frequency spectrum. The purpose of the music includes: 1) to put a subject in a relaxed state of awareness/mindfulness, and in a position of openness to therapy; and 2) to introduce the 40 Hz signal to the subject to initiate targeted frequency tuning in the subject's brain.

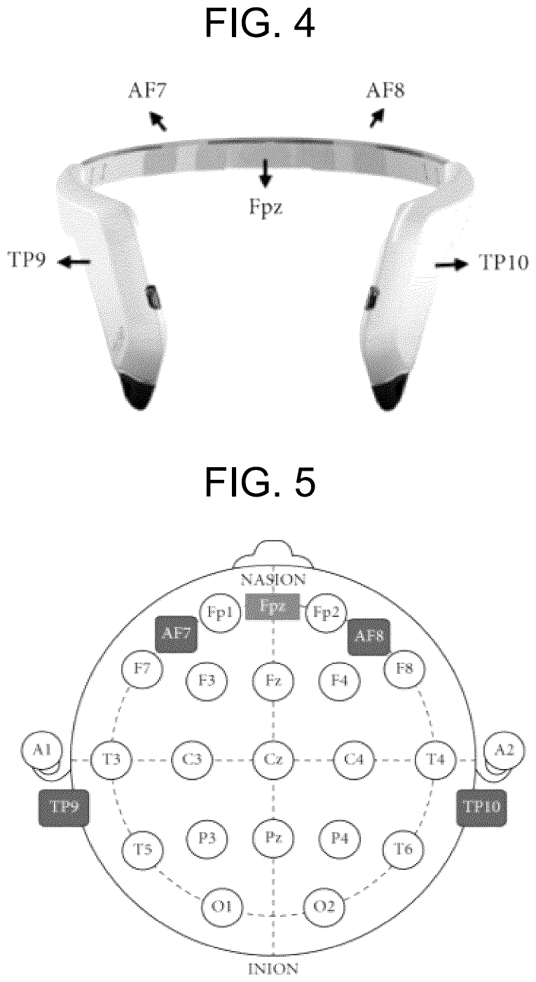

FIG. 4 illustrates a Muse EEG Headband that can be worn around the subject's head to obtain results of the applied composition of music to the subject's brain. The Muse EEG Headband reads EEG brain frequencies in four general brain regions: the left and right sides of the tempo-parietal region (tP9, TP10), and the left and right regions in the anterior-frontal region (AF7, AF8). The subject will preferably wear ear buds to receive the composed music specifically designed for a 40 Hertz frequency spectrum.

FIG. 5 illustrates the regions of the brain in which the EEG measurements are obtained using the Muse EEG Headband.

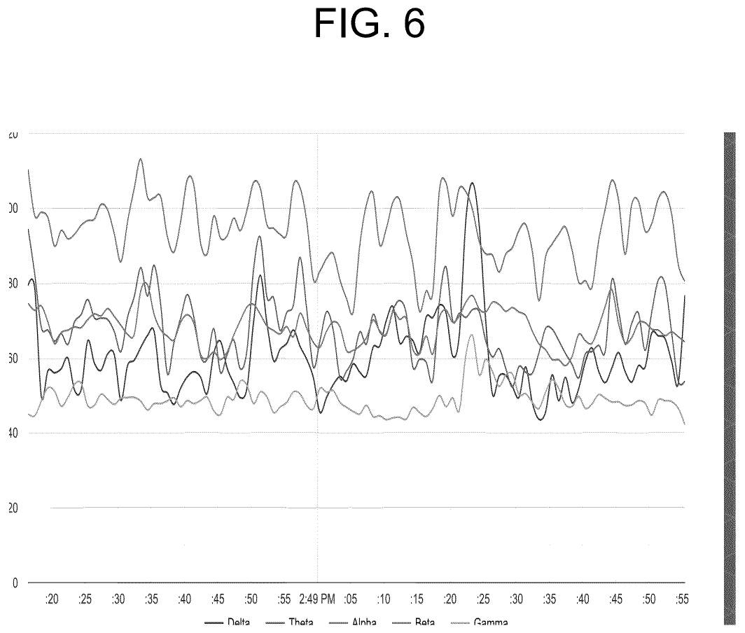

FIG. 6 illustrates a graph of frequency vs. time where brain waves are detected in response to music played in the 40 Hertz frequency spectrum. More specifically, a muse headband was placed on a subject to detect brain waves in response to composed music specifically designed for a 40 Hertz frequency spectrum. The vertical axis represents frequency from 0 to 120 Hz. The horizontal axis represents time in seconds. As illustrated, the marking of time:25 to the right of the time labeled 2:49 PM shows where several of the brain waves approach the 40 Hz target. The red line represents Delta, the purple line represents Theta, the blue line represents Alpha, the green line represents Beta and the orange line represents Gamma.

(40 Hz), roughly low E on a piano is the fundamental frequency upon which the specifically designed composition of music is based. In addition, random rhythmic pulsations can be employed as well as silences for resetting the subject's attention. A spectral wavetable synthesizer can be employed to play a minimalistic melody employing simple intervals in triadic movement. This specific melodic device of ascending and descending is mimicking the expansion and contraction of the myelin sheath as well as the building up of action potentials, which is the underlying mathematical model of this application. This is the translation of this concept into a musical composition. This specifically designed music is not meant to stir the memory by association, as that is a subjective endeavor, but instead is designed to enable the listener to participate fully in the benefits of the moment of brain tuning.

A preliminary implementation of the inventive process is based on the following considerations:
1) the optimum frequency or frequencies of resonant forces is created by neuroelectric tuning of apical dendrites in loop circuits with the thalamus through stimulations from appropriate music, light, sound, mechanical vibrations or combinations thereof,
2) Estimate by EEG or Brain Magnetic Stimulation (BMS) methods (during external excitation by sound, music, or light flicker), the possible resonant frequencies of the corticothalamic loops;
3) Determine the optimum resonant frequency based on the action potential time pulse width and the maximum number of pulses per second without overlap in time from adjacent action potentials in the train of action potentials. For example, 40 Hz nearly satisfies this requirement;
4) Create a train of action potential pulses or spikes of equal spacing in time and space in the appropriate neuronal structure of the cerebral cortex by resonance behavior;
5) Determine the extent of neuron synchrony of resonant action potentials in the cerebral cortex by EEG and/or MPS measurements; and
6) Using the appropriate frequency spectrums of music, light flicker, etc., create the required electrical oscillation in the appropriate part of the subject's brain having diseased neurons (possibly verify by MRI) for plaque treatment by mechanical and electrostatic forces.

During the testing of a subject, first, the lighting was dimmed, and ambient sound was mitigated as much as possible in order to facilitate the process for optimal effect. During testing neural firing (Action Potentials) of 40 Hz at regular intervals was experimentally observed using an iworx214 EEG and Lab scribe v4 software. A sense of peace was felt by the subjects during their time of listening to the music as well as experiencing visual perceptions of a green/yellow form and a deep blue and purple color. After the treatment a sense of clarity and well-being remained for many hours and could be recalled the following day by the subject.

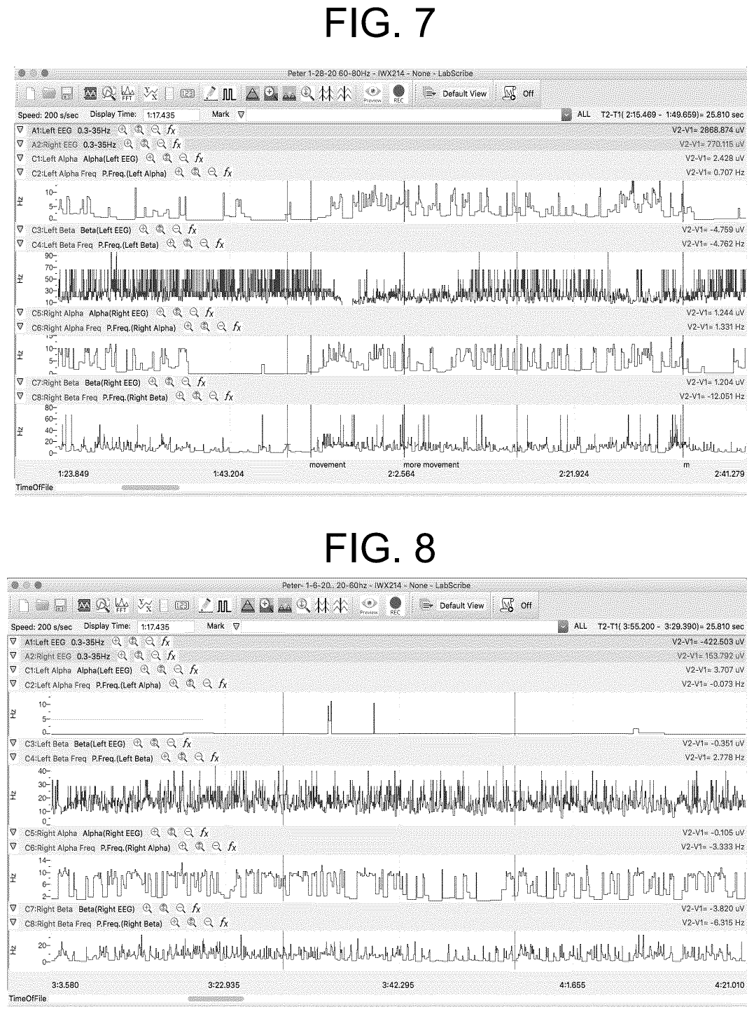

FIG. 7 illustrates observations of neural firing (Action Potentials) from a 60-80 Hz session at regular intervals using an iworx214 EEG and Lab scribe v4 software.

FIG. 8 illustrates observations of neural firing (Action Potentials) from a 40 Hz session at regular intervals using an iworx214 EEG and Lab scribe v4 software.

The special music designed for the brain tuning experiments demonstrated the necessary 40 Hz action potential waveforms as observed in the EEG recorded data.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A generator of music comprising the appropriate frequency components to excite the resonant action potentials in the cerebral cortex of a human's brain for compression of the diseased plaque associated brain neurons.

2. The generator according to claim 1, wherein the appropriate frequencies are applied and positioned outside the brain to achieve an overall sensory activity for enhancement of the treatment volume of diseased brain neurons.

3. The generator according to claim 1, wherein reduction of brain AD is detected by TMS measurement of the eye retina.

4. A generator to generate a blinking light for a human eye in the visual spectrum of appropriate color, intensity and pulse rate to excite the resonant action potentials in the cerebral cortex for the compression of the diseased plaque associated with brain neurons.

5. A generator of electromagnetic waves over a broad range of oscillations that can include electric or magnetic energy separately applied such as with an antenna or commercially available TMS device (trans magnetic stimulation) to excite the resonant action potentials in the cerebral cortex for the compression of the diseased plaque associated with brain neurons.
